(19) United States
(12) Patent Application Publication
(10) Pub. No.: US 2021/0285946 A1
(43) Pub. Date: Sep. 16, 2021

(54) STRIP DETECTING APPARATUS

(71) Applicant: National Cheng Kung University, Tainan City (TW)

(72) Inventors: Yu-Cheng Lin, Tainan City (TW); Wei-Chien Weng, Chiayi County (TW); Kai-Wen Lin, Tainan City (TW)

(21) Appl. No.: 16/931,468

(22) Filed: Jul. 17, 2020

(30) Foreign Application Priority Data
Mar. 13, 2020 (TW) 109108399

(51) Int. Cl. G01N 33/543; G01N 21/77; B01L 3/00; G06T 7/00; G06T 7/80; G06T 7/70; G06K 19/06; G06K 7/14; G06K 7/10

(52) U.S. Cl. CPC G01N 33/54386; G01N 21/77; B01L 3/5023; G06T 7/0012; G06T 7/80; G06T 7/70; G06T 2207/30004; G06K 7/1417; G06K 7/10722; G01N 2201/062; G01N 2201/127; G01N 2201/08; B01L 2300/0825; G06K 19/06037

(57) ABSTRACT

A strip detecting apparatus includes a black box, a light guide plate disposed on the black box, a light source, an optical calibration platform, a strip platform, a test strip contained in the strip platform, an image capturing device and an image processing device. The light source is disposed on the black box to emit light toward the black box via the light guide plate. The image capturing device is disposed on the black box for respectively capturing an optical calibration image and a detection image when the optical calibration platform and the strip platform are inserted into the black box respectively. The image processing device is coupled to the image capturing device for calibrating relationship information between the concentration and the grayscale value of the test strip according to the optical calibration image and for generating a test result according to the calibrated relationship information and the detection image.

### BACKGROUND OF THE INVENTION

### 1. Field of the Invention

The present invention relates to a strip detecting apparatus, and more specifically, to a strip detecting apparatus utilizing a black box to perform optical calibration via an optical calibration platform and generate a test result for a test strip.

### 2. Description of the Prior Art

With development of medic detection technology, various kinds of test strips are commonly applied to daily life, such as a urine analysis test strip, a fecal occult blood test strip, and a helicobacter pylori test strip, so as to help a user clearly know his health condition. A conventional test method involves utilizing a test strip (e.g. a urine analysis test strip often inspection items or a lateral flow immunoassay test strip) to be in contact with a sample and then comparing colors presented on the test strip with color scale patterns on a colorimetric plate for determining a test result.

However, the prior art usually adopts a visual inspection method to determine a test result. This method not only results in a time-consuming and strenuous manual colorimetric process, but also causes a colorimetric error problem easily. Although image identification technology has been developed to replace the aforesaid visual inspection method for obtaining a more precise test result, image shift, image tilt, capturing angle tilt, or image shake may easily occur since the user needs to take photographs of the colorimetric plate and the test strip by himself, so as to greatly influence accuracy of the test result. Thus, how to provide a strip detecting apparatus for helping a user complete a strip detecting process quickly and providing a precise test result is an important issue in medic rapid test.

### SUMMARY OF THE INVENTION

The present invention provides a strip detecting apparatus. The strip detecting apparatus includes a black box, a light guide plate, a light source, an optical calibration platform, a strip platform, a test strip, an image capturing device, and an image processing device. The black box has a chamber and an insertion opening. The light guide plate is disposed above the chamber. The light source is disposed on the black box to emit light into the light guide plate and toward the black box via the light guide plate. The optical calibration platform has a plurality of color scale patterns. The strip platform has a containing slot structure. The test strip is contained in the containing slot structure. The test strip reacts with a sample to generate at least one color block. The image capturing device is disposed on the black box for respectively capturing an optical calibration image and a detection image when the optical calibration platform and the strip platform are inserted into the black box respectively via the insertion opening. The image processing device is coupled to the image capturing device. Relationship information between the concentration and the grayscale value of the test strip is saved in the image processing device. The image processing device calibrates the relationship information according to images corresponding to the plurality of color scale patterns in the optical calibration image and generates a test result according to the calibrated relationship information and an image corresponding to the color block in the detection image.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is an assembly diagram of a strip detecting apparatus according to an embodiment of the present invention.

FIG. 2 is an exploded diagram of the strip detecting apparatus in FIG. 1.

FIG. 3 is a top view of an optical calibration platform according to an embodiment of the present invention.

FIG. 4 is a top view of a test strip in FIG. 2 being contained in a strip platform.

FIG. 5 is a top view of a quick response code platform according to an embodiment of the present invention.

### DETAILED DESCRIPTION

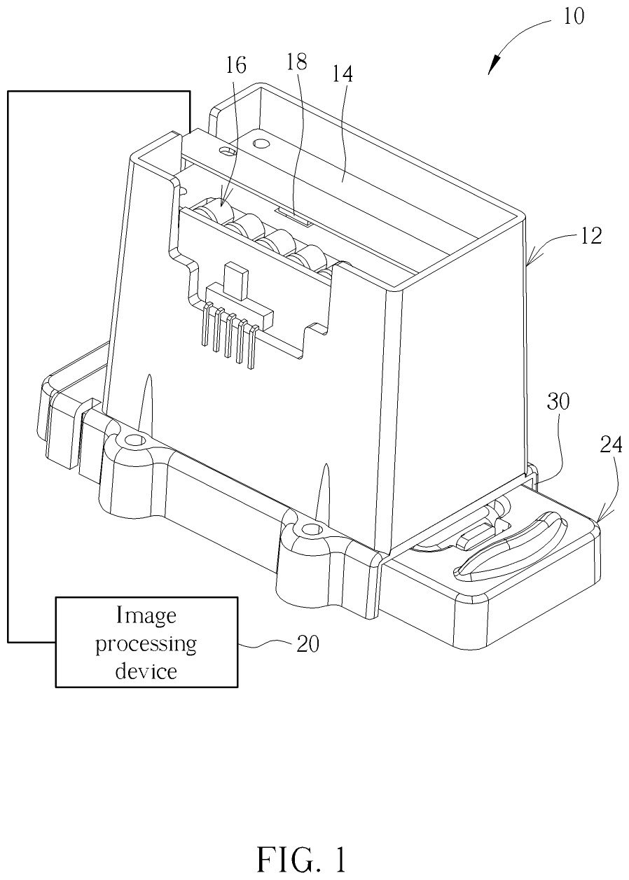

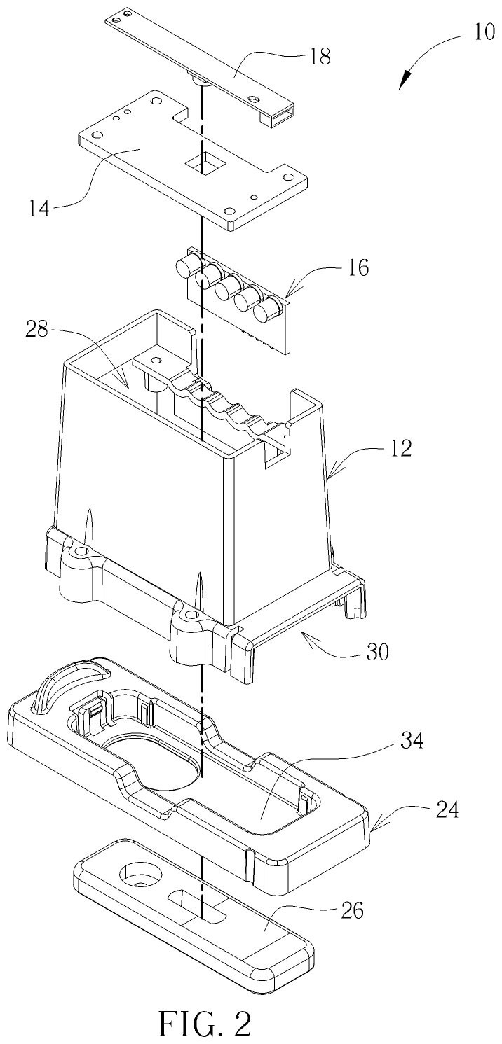

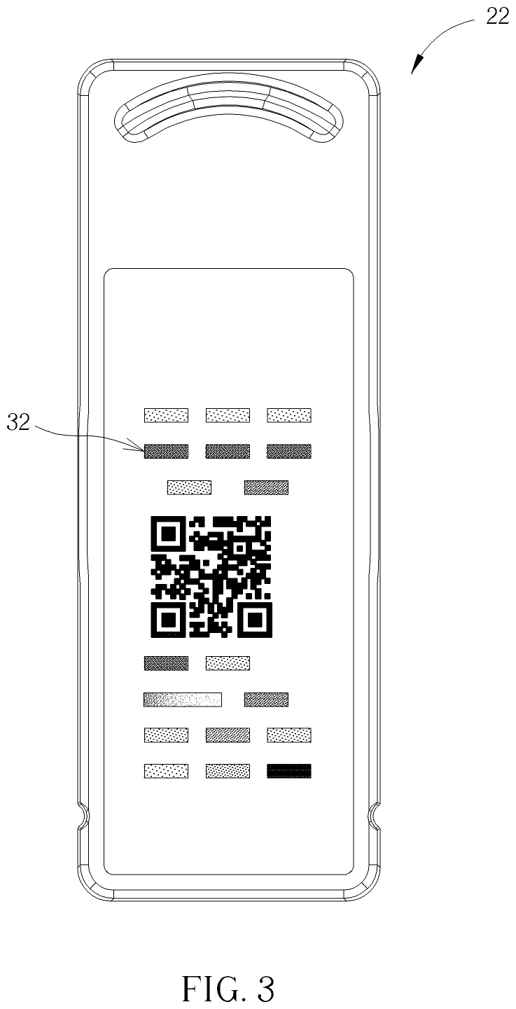

Please refer to FIG. 1, FIG. 2, and FIG. 3. FIG. 1 is an assembly diagram of a strip detecting apparatus 10 according to an embodiment of the present invention. FIG. 2 is an exploded diagram of the strip detecting apparatus 10 in FIG. 1. FIG. 3 is a top view of an optical calibration platform 22 according to an embodiment of the present invention. As shown in FIG. 1, FIG. 2, and FIG. 3, the strip detecting apparatus 10 includes a black box 12, a light guide plate 14, a light source 16, an image capturing device 18, an image processing device 20 (briefly depicted by a functional block in FIG. 1), the optical calibration platform 22, a strip platform 24, and a test strip 26. The black box 12 has a chamber 28 and an insertion opening 30 for providing a darkroom environment. The light guide plate 14 is disposed above the chamber 28. The light source 16 could be preferably composed of light emitting diodes (but not limited thereto) and includes at least one of a visible light source device and an invisible light source device (e.g. the light source 16 could be a white light source device for performing visible light detection or an ultraviolet light source device for performing invisible light detection, or the light source 16 could include a white light source device and an ultraviolet light source device for selectively providing visible light or invisible light according to the user operations) . The light source 16 is disposed on the black box 12 to emit light into the light guide plate 14 and toward the chamber 28 via optical guidance of the light guide plate 14 (the related description for the light guide design of the light guide plate 14 is commonly seen in the prior art and omitted herein) for providing sufficient and uniform light during the detecting process.

The image capturing device 18 could be preferably a network camera (but not limited thereto) an disposed on the black box 12 to be aligned with the chamber 28 for image capturing to perform subsequent optical calibration and detection. The image processing device 20 could be preferably a circuit board having an image processing function, such as a Raspberry Pi circuit board (but not limited thereto) . The image processing device 20 is connected to the image capturing device 18 and has relationship information between the concentration and the grayscale value of the test strip 26 (could be a fitting curve of the concentration and the grayscale value of the test strip 26 according to practical experience, but not limited thereto) saved therein. As such, the image processing device 20 can generate a test result according to the image transmitted from the image capturing device 18 and the aforesaid relationship information.

In practical application, for preventing wrong determination of the strip detecting apparatus 10 due to malfunction, aging or decay of the light source 16 (or wrong determination caused by the light sources and the darkroom environments provided by the strip detecting apparatuses being different from each other), the strip detecting apparatus 10 can utilize the optical calibration platform 22 to perform optical calibration before the strip detecting process. In this embodiment, the optical calibration platform 22 adopts the color scale pattern design. For example, as shown in FIG. 3, the optical calibration platform 22 could have a plurality of color scale patterns 32 (the amount and the pattern arrangement design of the color scale patterns 32 could depend on the practical application of the strip detecting apparatus 10 and could be not limited to FIG. 3) . As such, a user can insert the optical calibration platform 22 into the chamber 28 via the insertion opening 30 and operate the image capturing device 18 to capture corresponding optical calibration image for subsequent optical calibration.

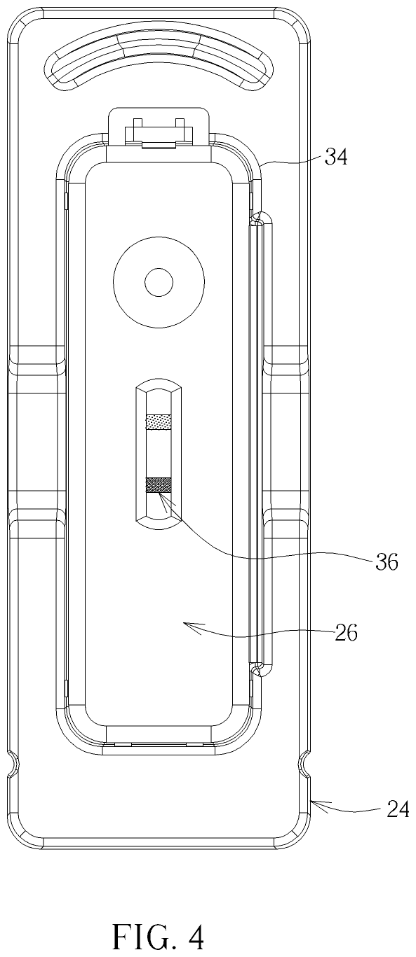

Please refer to FIG. 2 and FIG. 4. FIG. 4 is a top view of the test strip 26 in FIG. 2 being contained in the strip platform 24. As shown in FIG. 2 and FIG. 4, the strip platform 24 could have a containing slot structure 34. The test strip 26 is contained in the containing slot structure 34 and generates at least one color block 36 (two shown in FIG. 4, but not limited thereto, meaning that the amount of the color block 36 depends on the practical application of the strip detecting apparatus 10) after being reacted with a sample. To be more specific, in this embodiment, the test strip 26 could be preferably a lateral flow immunoassay test strip for performing a fecal occult blood test (but not limited thereto, meaning that the present invention can be applied to all the strip designs for color test (e.g. a urine analysis test strip of ten inspection items) to provide a multi-category test function). For example, when a lateral flow immunoassay test is performed, the color block 36 composed of a control line and a result line in FIG. 4 is shown on the test strip 26 if the test result is positive. On the contrary, the color block 36 composed of one single control line is shown on the test strip 26 if the test result is negative. The color block design is not limited to the aforesaid embodiment. For example, in another embodiment, the present invention could adopt the design that the color block 36 composed of one single result line is shown on the test strip 26 if the test result is negative. To be noted, the control line shown on the test strip 26 means that the reagent and the test operation of the test strip 26 are effective, and different colors of the result line represent different fecal occult blood concentrations.

Via the aforesaid design, when the user wants to perform the test operation of the strip detecting apparatus 10, the user just needs to insert the optical calibration platform 22 into the chamber 28 via the insertion opening 30 such that the image capturing device 18 can capture a corresponding optical calibration image. At this time, the image processing device 20 can compare an image corresponding to the plurality of color scale patterns 32 in the optical calibration image captured by the image capturing device 18 with the original grayscale values corresponding to the plurality of color scale patterns 32 to generate a comparison result, and can calibrate the relationship information between the concentration and the grayscale value of the test strip 26 according to the comparison result, so as to prevent the strip detection of the strip detecting apparatus 10 from being influenced by light brightness variation. In such a manner, the present invent invention can efficiently improve accuracy of the strip detecting apparatus 10.

After the aforesaid optical calibration process is completed, the user can place the test strip 26, which has been reacted with a sample to generate the color block 36, into the containing slot structure 34 of the strip platform 24, and then can insert the strip platform 24 into the chamber 28 via the insertion opening 30 (as shown in FIG. 1), so that the image capturing device 18 can capture a corresponding test image. At the same time, the image processing device 20 can generate a corresponding test result according to the calibrated relationship information and an image corresponding to the color block 36 in the test image. For example, the image processing device 20 could perform grayscale conversion on the test image and then perform image cutting on the test image to generate a desired analysis region and obtain a region background value. Subsequently, the image processing device 20 could analyze the desired analysis region according to a waveform algorithm to determine whether the control line and the result line exist and obtain corresponding practical grayscale values. Finally, the image processing device 20 could compare the practical grayscale values of the control line and the result line with the calibrated relationship information, so as to precisely determine the fecal occult blood concentration and complete the test operation of the strip detecting apparatus 10.

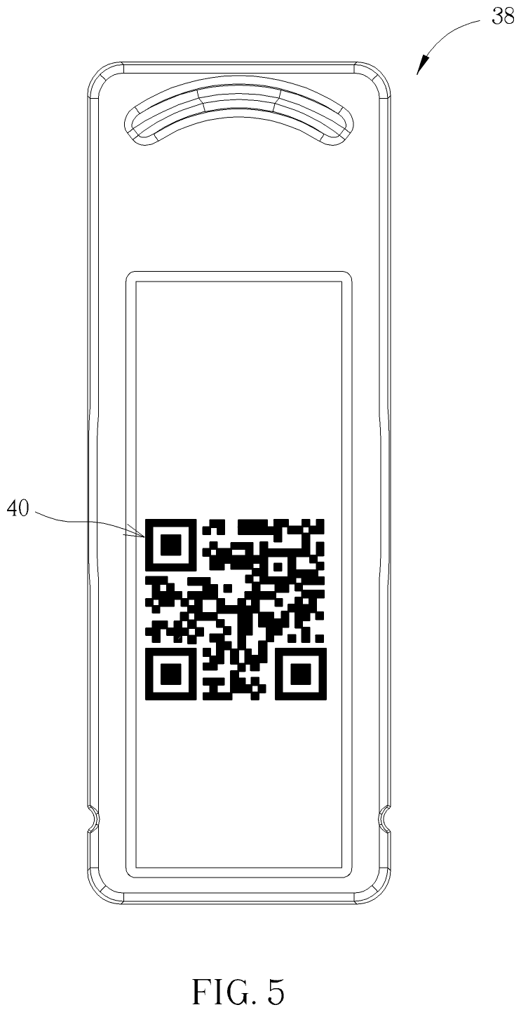

To be noted, since the image processing device 20 obtains the practical grayscale value of the color block 36 via the aforesaid optical comparison process, the image processing device 20 can still determine the correct fecal occult blood concentration according to the practical grayscale value even if the concentration is low or equal to zero. For improving the computing speed and making the test result convenient to store, in another embodiment, the image processing device 20 could be a network server. Accordingly, the present invention can upload the test image to the network server for processing the image by cloud computing to generate the test result, and then can store the test result transmitted from the network server to a database of a related application for a user to browse the test result conveniently. In practical application, for preventing wrong detection and further improving the computing speed, as shown in FIG. 5, the strip detecting apparatus 10 could further include a quick response code platform 38. The quick response code platform 38 could have a quick response code pattern 40. Accordingly, before performing the aforesaid strip detecting process, the user can insert the quick response code platform 38 into the chamber 28 via the insertion opening 30 such that the image capturing device 18 can capture and then transmit a corresponding quick response code image to the image processing device 20 for decoding the quick response code image, so as to obtain the related detection information (e.g. inspection item, detecting amount, detecting category, reaction time, calibration curve parameter, or detecting threshold) of the test strip 26 quickly and precisely.

In summary, compared with the prior art, the present invention utilizes the black box to perform optical calibration via the optical calibration platform inserted therein and determine the test result of the test strip on the strip platform inserted therein. In such a manner, the strip detecting apparatus of the present invention not only obtains the test result quickly without a complicated and time-consuming detecting process, but also allows that the user can just insert the test strip into the black box for detection of the test strip without performing an additional strip positioning process, so as to solve the prior art problem that image shift, image tilt, capturing angle tilt, or image shake easily occurs since the user needs to take photographs of the colorimetric plate and the test strip by himself. Thus, the present invention can efficiently prevent wrong detection of the test strip and greatly improve the detecting accuracy of the strip detecting apparatus.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A strip detecting apparatus comprising:
a black box having a chamber and an insertion opening;
a light guide plate disposed above the chamber;
a light source disposed on the black box to emit light into the light guide plate and toward the black box via the light guide plate;
an optical calibration platform having a plurality of color scale patterns;
a strip platform having a containing slot structure;
a test strip contained in the containing slot structure, the test strip reacting with a sample to generate at least one color block;
an image capturing device disposed on the black box for respectively capturing an optical calibration image and a detection image when the optical calibration platform and the strip platform are inserted into the black box respectively via the insertion opening; and
an image processing device coupled to the image capturing device, relationship information between the concentration and the grayscale value of the test strip being saved in the image processing device, the image processing device calibrating the relationship information according to images corresponding to the plurality of color scale patterns in the optical calibration image and generating a test result according to the calibrated relationship information and an image corresponding to the color block in the detection image.

2. The strip detecting apparatus of claim 1, wherein the light source comprises at least one of a visible light source device and an invisible light source device.

3. The strip detecting apparatus of claim 1, wherein the strip detecting apparatus further comprises a quick response code platform having a quick response code pattern, and the image capturing device captures a quick response code image when the quick response code platform is inserted into the chamber via the insertion opening and decodes the quick response code image to obtain related test information of the test strip.
